(19) United States
(12) Patent Application Publication
(10) Pub. No.: US 2005/0014174 A1
(43) Pub. Date: Jan. 20, 2005

(54) Method for the detection of nucleic acids

(75) Inventors: Jens Burmeister, Koln (DE); Edgar Diessel, Koln (DE)

Correspondence Address: NORRIS, MCLAUGHLIN & MARCUS, PA, 875 THIRD STREET, 18TH FLOOR, NEW YORK, NY, 10022, US

(73) Assignee: Bayer Technology Services GmbH, Leverkusen (DE)

(21) Appl. No.: 10/844,769

(22) Filed: May 12, 2004

(30) Foreign Application Priority Data
May 21, 2003 (DE) 103 22 912.4

(51) Int. Cl. C12Q 1/68

(52) U.S. Cl. 435/6

(57) ABSTRACT

A method is described for the specific detection of nucleic acids on a solid phase. The method involves the steps of: A) providing a surface having immobilized nucleic acids or nucleic-acid analogues, which are suitable for forming non-covalent (base-pair) bonds with the target nucleic acids, B) non-stringent hybridization of the target nucleic acids to be detected onto the immobilized nucleic acids from a solution of the analyte nucleic acid, C) labeling of the nucleic acids of the analysis mixture with labeling elements, D) optionally repeated treatment of the surface with a washing liquid in order to remove weakly bound nucleic acids, and E) detection of the nucleic-acid pairs remaining on the surface with the aid of the labeling unit bonded to them, by means of optical, optical-spectroscopic, electrical, mechanical or magnetic detection methods; wherein steps B) and C) can be carried out in any order.

The invention relates to a method for the specific detection of nucleic acids on a solid phase. The invention furthermore relates to kits which contain the reagents that are required for carrying out the described assays. The detection of deoxyribonucleic acids (DNA) or ribonucleic acids (RNA) has a wide range of application, for example in human and veterinary diagnosis, in the food industry, in environmental analysis, in crop protection, in biochemical or pharmacological research and in forensic medicine.

So-called DNA arrays, which permit the simultaneous analysis of a large number of different sequences, have become established as a standard method for the detection of nucleic acids. DNA arrays are-used, for example, for expression profiling, sequencing by hybridization, analysis of single nucleotide polymorphisms (SNPs) etc. Examples of the production and use of DNA arrays can be found, for example, in DNA Microarrays, D. Bowtell and J. Sambrook (eds.), Cold Spring Harbor Laboratory Press, New York 2003.

Nucleic acids or nucleic-acid analogues may be used as detection elements on DNA arrays. Examples of nucleic-acid analogues are PNA (M. Egholm et al., Nature 365, 566-568 (1993)), LNA (D. A. Braasch, D. R. Corey, Chem. Biol. 8, 1-7 (2001)) or nucleic acids modified on the sugar backbone (M. Shimizu et al., FEBS Letters 302, 155-158 (1992)).

DNA arrays can be read, for example, by optical, electrical, mechanical or magnetic methods.

Optical detection methods are based, for example, on the detection of fluorescence-labeled biomolecules on dielectric surfaces. The fluorescence may in this case be stimulated by means of planar optical waveguides (see U.S. Pat. No. 5,959,292), total reflection at interfaces (see. DE 196 28 002 A2), or on the surface of optical fibres (see U.S. Pat. No. 4,447,546).

Electrical biosensors rely, for example, on the detection of analytes which are labeled by metal particles, for example, nanoparticles. For detection, these particles are enlarged by autometallographic deposition until they short-circuit a microstructured electrical circuit. This is demonstrated by a straightforward DC resistance measurement (U.S. Pat. Nos. 4,794,089; 5,137,827; 5,284,748).

Field-effect transistors can be used as electronic transducers, for example, for an enzymatic reaction: Zayats et al. Biosens. & Bioelectron. 15, 671 (2000).

As mechanical transducers, quartz resonators are described in which the resonant frequency is varied by application of mass: Steinem et al., Biosens. & Bioelectronics 12, 787 (1997). In an alternative mechanical transducer, surface waves that are modified by target adsorption are stimulated using interdigital structures: Howe et al., Biosens. & Bioelectron. 15, 641 (2000).

If the target molecules are labeled with magnetic beads, then the recognition reaction can be detected by means of the magnetic effect of the beads on the giant magnetic resistance (GMR) of a corresponding resistor: Baselt et al. Biosens. & Bioelectron. 13, 731 (1998).

Labeling of the target nucleic acids hybridized onto the array by electrically conductive particles is advantageous, in particular, for the electrical method. WO 99/57550-A2 describes the labeling of nucleic-acid targets which have been hybridized onto immobilized nucleic acids between Planar electrode pairs, for example, with gold particles. By autometallographic enhancement of the gold particles, for example, with solutions of metal ions in the presence of reducing agents, it is possible to produce an electrically conductive, metal film between the electrode pairs, which generally consists of a network of particles electrically conductively connected to one another. The presence of the target is detected by detecting the conductance of the metal film. WO 99/57550-A2 describes that the labeling of the target may be carried out before or after the interaction with the immobilized detector elements. A preferred embodiment of WO 00/25136-A2 describes the labeling of an oligonucleotide with cis-platinum-biotin and subsequent labeling of the biotinylated target with a streptavidin-gold cluster. The gold-labeled target is hybridized onto immobilized nucleic acids between electrode pairs. A conductive gold film, similar to the silver film described above, is formed after autometallographic enhancement with gold salts in the presence of reducing agents. The hybridization event is indicated by measurement of the electrical contact between the electrode pairs.

Another preferred embodiment of WO 00/25136-A2 describes the reaction of a target molecule with biotin. The biotin-labeled target is bound to detector elements immobilized between electrode pairs. The bound biotinylated target is subsequently labeled with colloidal gold particles, which are linked with avidin or streptavidin. A conductive gold film is formed after autometallographic enhancement with gold salts in the presence of reducing agents. The bonding event is indicated by measurement of the electrical contact between the electrode pairs.

Nucleic acids which have been hybridized onto immobilized detector elements, and which have been labeled with gold particles, can also be detected by optical methods. WO 02/0281 0-A2 describes the formation of precipitates on array elements, and determination of the time profile of the precipitation in the form of signal intensities, for example, by optical or electrical methods. WO 02/02810-A2 claims that the targets are linked with, for example, colloidal metal particles before, during or after the interaction with the immobilized detector elements.

Two different methods for the detection of nucleic acids on microarrays, on the basis of labeling with gold particles, are commercially available at present. The ArrayTube System (Clondiag Chip Technologies) is based on the hybridization of, for example, targets which are amplified with biotinylated primers and are hybridized onto the chip under stringent conditions. The targets are subsequently labeled with streptavidin-gold (colloidal gold, 5 nm). The hybridization is optically detected after autometallographic enhancement with silver salts. The ArrayTube instruction manual expressly recommends that the gold labeling be carried out only after the stringent hybridization.

The detection system from Genicon Sciences, which is also commercially available, is based on the optical detection of gold particles which have been linked with anti-biotin antibodies. Autometallographic enhancement of the gold particles is not necessary for use of the resonance light scattering technology. In all the applications of RLS technology to nucleic acids which are described by Genicon Sciences, the labeling of the biotinylated targets is carried out only after the stringent hybridization onto the array.

In summary, it may be stated that the detection of nucleic acids on DNA microarrays by labeling with gold particles, which have, for example, been linked with streptavidin or antibodies, is preferably carried out according to the state of the art only after the hybridization of nucleic acids onto the DNA array and after having-performed the discrimination.

An alternative approach to the labeling of nucleic acids on DNA arrays with gold particles is based on the use of DNA-coated gold particles as labeling units. In this method, the DNA to be detected is detected in a sandwich-hybridization assay between an immobilized detector DNA, the target and a DNA-coated gold particle. No direct binding of gold to the nucleic acid to be detected takes place in this method; instead, the binding of a DNA-coated gold particle is brought about by an additional hybridization. The use of DNA-coated gold particles for the optical detection of nucleic acids on DNA microarrays has been described, for example, by Taton et al. (Taton et al., Science 2000, 289, 1757-1760). The same method has also been used for the electrical detection of nucleic acids on DNA microarrays, described e.g. in S.-J. Park et al., Science 2002, 295, 1503-1506. From the work into the labeling of nucleic acids hybridized onto DNA microarrays with DNA-coated gold particles, it is found that these particles increase the specificity of the discrimination of closely related DNA sequences by raising the melting points of the DNA sandwich complexes compared with native DNA sandwich complexes. This improves, in particular, the discrimination of single nucleotide polymorphisms (SNPs). Disadvantages of using DNA-coated gold particles involve the very elaborate production, the instability of the conjugates (if gold particles are coated with thiolated oligonucleotides, for example, then these conjugates are unstable with respect to higher-concentration salt solutions and with respect to higher temperatures, and are susceptible to agglomeration), the high production costs due to the large quantities of oligonucleotides required per gold particle and the dependency of the labeling on a specific hybridization.

The latter point should be emphasized, since the particular advantage of DNA arrays involves the simultaneous analysis of a large number of different sequences. If different genes are analysed next to one another, for example, then a sequence-specific detector is required for each gene in the case of a sandwich assay. This outlay significantly restricts the practicable multiplexability of a DNA array labeled with DNA-coated gold particles.

A fundamental problem with the detection of nucleic acids on DNA microarrays is how to simultaneously guarantee selectivity and sensitivity of the hybridization. A particularly high selectivity of the hybridization must be guaranteed whenever single nucleotide polymorphisms (SNPs) are being detected, for example, by allele-specific hybridization onto DNA arrays. An example of SNP detection by allele-specific hybridization onto DNA arrays is described in Iwasaka at al., DNA Research 2002, 9, 59-62. A particular problem with allele-specific hybridization is how to discriminate those base pairings, between the target and the immobilized sample, which differ only a little in their thermodynamic stability. Examples of base pairings with similar thermodynamic stability are GC and GT or GC and GG base pairs.

The selectivity of the allele-specific hybridization is achieved, according to the state of the art, either by stringent hybridization conditions or else by stringent washing steps after the first, non-stringent hybridization. The selectivity which can be achieved in this way with stringent hybridizations, however, reduces the absolute signals of the hybridization reaction for the “matching” (generally Watson-Crick) base pairs.

The reduction of the absolute signals due to stringent hybridization conditions on DNA arrays is a disadvantage, in particular, whenever a DNA array is intended to be labeled with gold, subsequently enhanced with silver and electrically read, since the likelihood of percolation paths being formed between gold colloids depends on the surface density of the colloids. In particular, for such a measurement method it is necessary to exceed a critical surface density, which constitutes a threshold value for the electrical conductance (D. Stauffer, A. Aharony: Percolation Theory—An Introduction, VCH, Weinheim, 1995, pp. 95 ff.).

The methods described above for the hybridization of nucleic acids onto DNA microarrays and labeling with gold particles have a number of disadvantages; especially whenever electrical reading is subsequently intended to take place. In methods mentioned according to the prior art for labeling with streptavidin- or antibody-coated gold particles, for example, the problem of the reduction of the absolute signals after stringent hybridizations is not resolved. Although methods which employ DNA-coated gold particles for the labeling of DNA arrays allow simultaneously sensitive and specific detection of closely related nucleic acids, they are nevertheless restricted to sandwich-based assays.

It is an object of the invention to improve the labeling of DNA arrays with labeling units, so that the surface density of the labeling units is increased while preserving the specificity of the hybridization. The object is in particular, to increase significantly the quantity of nucleic-acid molecules to be detected which have been hybridized onto the chip, while maintaining the selectivity of the hybridization.

This object is achieved according to the invention in that nucleic-acid targets are hybridized non-stringently onto detector nucleic acids immobilized on DNA arrays, the labeling of the nucleic-acid targets hybridized onto the detector nucleic acids with labeling units is carried out before or after this hybridization step, and the discrimination is subsequently carried out. The discrimination of different labeled sequences is carried out, for example, by stringent washing steps. Surprisingly, it has been found that gold labeling, for example, leads to a significant increase in the temperature which is required for discrimination of different nucleic-acid sequences by stringent washing steps. The discrimination of closely related sequences is improved significantly by the described method.

The invention relates to a method for the detection of target nucleic acids from a mixture of different nucleic acids, with the steps of
A) providing a surface having immobilized nucleic acids or nucleic-acid analogues, which are suitable for forming non-covalent (base-pair) bonds with the target nucleic acids,
B) non-stringent hybridization of the target nucleic acids to be detected onto the immobilized nucleic acids from a solution of the analyte nucleic acid,
C) labeling of the nucleic acids of the analysis mixture with labeling units,
D) optionally repeated treatment of the surface with a washing liquid in order to remove weakly bound nucleic acids, and
E) detection of the nucleic-acid pairs remaining on the surface with the aid of the labeling unit bonded to them, by means of optical, optical-spectroscopic, electrical, mechanical or magnetic detection methods.

In an alternative method, steps B and C may be interchanged.

The invention therefore also relates to a method for the detection of target nucleic acids from a mixture of different nucleic acids, with the steps of
A) providing a surface having immobilized nucleic acids or nucleic-acid analogues, which are suitable for forming non-covalent (base-pair) bonds with the target nucleic acids,
B′) labeling of the nucleic acids of the analysis mixture with labeling units,
C′) non-stringent hybridization of the labeled nucleic acids onto the immobilized nucleic acids,
D) optionally repeated treatment of the surface with a washing liquid in order to remove weakly bound nucleic acids, and
E) detection of the nucleic-acid pairs remaining on the surface with the aid of the labeling unit bonded to them, by means of optical, optical-spectroscopic, electrical, mechanical or magnetic detection methods.

Stringent washing steps may preferably be carried out by washing the DNA array with thermally regulated buffer solutions, the temperature of the buffer solutions lying above the temperature used for the hybridization of the analyte nucleic acid onto the immobilized nucleic acids.

The person skilled in the art knows that the temperature at which half of streptavidin becomes denatured is 75° C. When all the binding pockets of streptavidin are saturated by biotin, there is a significant stabilization with respect to thermally induced denaturing (M. Gonzalez at al. Biomol. Eng. 16, 67-72 (1999)). If gold particles are coated with streptavidin and subsequently bound to biotinylated targets, saturation of the streptavidin molecules with biotin does not generally take place. The person skilled in the art may therefore assume that streptavidin-coated gold particles are not stable with respect to temperatures above 60-70° C, but that denaturing of the protein instead leads to coagulation of the gold particles. According to the prior art, the person skilled in the art will therefore avoid carrying out washing steps at temperatures above 60-70° C. after having labeled nucleic acids hybridized onto arrays, for example with streptavidin-coated gold particles.

The present invention is based on the observation that, for example, streptavidin-coated gold particles have a sufficient thermal stability in order for stringent washing steps to be carried out on arrays after labeling of the hybridized nucleic acids.

Stringent washing steps may also preferably be carried out by washing the DNA array with buffer solutions, the ionic strength of which lies below the ionic strength of the buffer solution used for the hybridization onto the immobilized nucleic acids. According to the invention, the two methods may also be arbitrarily combined in order to adjust the stringency.

A preferred alternative of the method is characterized in that the stringent washing steps are carried out with sodium-chloride buffer solutions, the concentration of the buffer solution lying below the sodium-chloride concentration selected for the hybridization onto the immobilized nucleic acids.

According to the invention, the target nucleic acids are labeled with labeling units. The binding between the nucleic acid and the labeling unit may be carried out using covalent bonds, coordination bonds or non-covalent bonds. For binding of the labeling units, the target nucleic acid needs to be functionalized with ligand molecules which in turn bind to ligand-binding receptor molecules with which the surface of the labeling units has been coated. Suitable receptor-ligand pairs are known to the person skilled in the art. Examples of receptor-ligand pairs are biotin-streptavidin or antibody-antigen.

An interaction between avidin, neutravidin or streptavidin as the receptor and biotin as the ligand is preferably selected as the receptor-ligand interaction.

Alternatively, the receptor-ligand interaction will also preferably be an interaction between an antibody and its antigen as the ligand.

The linking of the target nucleic acid with ligands is particularly preferably carried out by enzymatic or chemical methods or by intercalation.

The functionalization of the nucleic acids with ligands is carried out by methods known to the person skilled in the art. Examples of suitable functionalizations are the use of modified nucleotides in enzymatic reactions such as PCR, primer extensions, transcription reactions or the use of ligand-coupled primers in enzymatic reactions such as PCR or primer-extension reactions. The binding of ligands to nucleic acids may also be carried out, for example, with intercalating molecules and (photo)chemical reactions between the nucleic acid and suitable ligands.

The labeling units are modified by coupling to receptor molecules, so as to enable binding to the ligands with which the nucleic-acid to be detected has been linked. Examples of such couplings are the coating of colloidal gold particles with streptavidin or antibodies.

The labeling units are selected so that they can be read by means of optical, optical-spectroscopic, electrical, mechanical or magnetic methods. The labeling units furthermore have properties which modify the release profile of the target nucleic acids hybridized onto the DNA array, so that the stringency (for example with respect to temperature, ionic strength) required for release of the labeled target nucleic acid is greater than the stringency required for the release of an unlabeled target nucleic acid.

Nanoparticles, metal complexes and/or clusters of materials such as Au, Ag, Pt, Pd, Cu, C etc. may preferably be used as labeling units. Further preferred examples of labeling units are beads, metal-coated beads, carbon nanotubes, proteins or other molecules with a molecular weight of preferably >10,000 g/mol and a particle size of preferably from 1 nm to about 10 μm.

A method which is characterized in that the surface has a set of different immobilized nucleic acids or nucleic-acid analogues is also preferred.

The DNA arrays labeled with the labeling units may be read by means of optical, electrical, mechanical or magnetic methods.

A method in which the nucleic acid to be detected is linked with biotin as a ligand, and the labeling is carried out using gold particles coated with avidin, neutravidin or streptavidin as a receptor, is particularly preferred.

A variant in which the nucleic acid to be detected is linked with an antigen, and the labeling is carried out using gold particles coated with antibodies, is also particularly preferred.

The labeling units may be enhanced before or during the reading (step E). A suitable enhancement reaction is, for example, the autometallographic enhancement of metal colloids with, for example, Au- or Ag-based enhancement solutions.

The invention furthermore relates to the use of the method according to the invention for the expression profiling of ribonucleic acids, or for the analysis of single point mutations (SNPs) and for the analysis of amplified genes:
Expression Profiling: Hybridization of the mixture of RNA analytes or of the corresponding DNA-mixture obtained after enzymatic reactions with the mixture of RNA analytes (e.g. cDNA) under non stringent conditions. Subsequently coupling of the RNA or DNA mixture to labeling entities before the application of stringent washing steps.
SNPs: Hybridization of the DNA-mixture containing SNPs or of the corresponding DNA-mixture obtained after enzymatic reactions with the DNA mixture under non stringent conditions. Subsequently coupling of the DNA mixture to labeling entities before the application of stringent washing steps.
Detection of amplified genes: Hybridization of the DNA-mixture containing amplified and/or non amplified genes or of the corresponding DNA-mixture obtained after enzymatic reactions with the DNA mixture under non stringent conditions. Subsequently coupling of the DNA mixture to labeling entities before the application of stringent washing steps.

The method according to the invention has the following advantages over the methods known from the prior art for the detection of nucleic acids on DNA arrays by means of labeling elements:
The discrimination of closely related nucleic acids by hybridizations is improved, the signal intensity achieved in the case of non-stringent hybridization being preserved for the hybridization reaction to be selected.
The linking of the target nucleic acid may be carried out before or after the non-stringent hybridization.
The labeling units are coupled to the target nucleic acid by a means other than hybridization, and therefore independently of the sequence of the target.
A large selection of different labeling units, which can be used for the labeling, is known to the person skilled in the art. This makes it possible to read DNA arrays by various methods.

The invention will be explained in more detail below with reference to exemplary embodiments.

### EXAMPLES

### Example 1

### Comparison of Discrimination Before and After the Labeling

2 DNA chips were prepared by immobilizing 5′-amino-modified, allele-specific oligonucleotides covalently on oxidized silicon chips, which were coated beforehand with polymers containing amine groups. The covalent immobilization was carried out by means of the homobifunctional cross-linker BS3 (bis-sulfo-succinimidyl suberate, from Pierce). The sequences of the allele-specific oligonucleotides were: 5′-amino- ttt ttt ttt cct aac tcg aac cc (SEQ ID NO: 1) (C sample) and 5′-amino- ttt ttt ttt cct aac ttg aac cc (SEQ ID NO: 2) (T sample). The chips had a size of 1 cm². The allele-specific oligonucleotides were immobilized on the chip surface in 5 μl duplicates, so that 4 spots were obtained per chip.

The DNA from a patient who had the CETP (cholesteryl ester transferase protein gene)-TaqIB genotype AA was amplified by PCR using standard methods, a biotinylated primer and a non-biotinylated primer being used. The biotin primer had the sequence 5′-biotin- ttg tgt ttg tct gcg acc (SEQ ID NO: 3), and the sequence of the non-biotinylated primer was 5′-ccc aac acc aaa tat aca cca (SEQ ID NO: 4).

The biotinylated strand of the PCR product had the sequence:

5′-biotin- tt gtgtttgtct gcgacccaga atcactgggg ttcAagttag ggttcagatc tgagccaggt tagggggtta atgtcagggg gtaaagatta ggaggttggt gtatatttgg tgttggg (SEQ ID NO: 5); A=SNP position.

2×50 μl of the PCR product were demineralized by centrifuging in Microcon-3 columns (from Millipore, MWCO=3,000). The demineralized PCR products were dissolved in 45 μl of 0.11 N NaOH, 0.9 M NaCl, 0.005% SDS. The alkaline target solutions were applied to the two identically prepared DNA chips and incubated for 10 min at 25° C.

The alkaline hybridization solution was neutralized by adding 5 μl of 1 M NaH₂PO₄, 1 M NaCl, 0.005% SDS. The chips with the hybridization solutions were incubated for 15 h at 25° C. in a humid chamber (=non-stringent hybridization).

100 μl of a solution of streptavidin-gold (10 nm, Sigma) were centrifuged for 30 min at 21,000 g. The supernatant was removed and the residue was taken up in 0.1 M phosphate buffer (pH 8.2), 1 M NaCl, 0.005% SDS (=buffer A).

After the end of the non-stringent hybridizations, the chips were washed with buffer A and subsequently-dried at 25° C.

Chip 1: Chip 1 was incubated for 2 h with 50 μl of the streptavidin-gold solution at 25° C. This was followed by discrimination of the alleles under stringent conditions. The discrimination was carried out by washing the chip for 5 min with preheated buffer A at 75° C. Buffer A was removed and washing was carried out with 0.1 M phosphate buffer (pH 8.2), 1 M NaNO₃, 0.005% SDS in order to remove interfering chloride ions before the silver enhancement.

Chip 2: Chip 2 was washed for 5 min with preheated buffer A at 55° C. Incubation was then carried out for 2 h with 50 μl of the streptavidin-gold solution at 25° C. Before the silver enhancement, washing was carried out at 25° C. with 0.1 M phosphate buffer (pH 8.2), 1 M NaNO₃, 0.005%.SDS.

For the silver enhancement of both chips, a solution was prepared by mixing one part of an aqueous 0.012 M AgNO₃ solution and four parts of an aqueous solution of 0.05 M hydroquinone and 0.3 M sodium citrate buffer (pH 3.8). The chips were immersed in this solution for 30 min. The silver enhancement was ended by washing the chips with water.

The DC resistance measurement of the silver-enhanced chip surfaces was carried out between externally applied electrodes. In order to compensate for inhomogeneities on the sample, 25 measurements per DNA spot were carried out at different positions of the spot using an automated measuring apparatus. The essential parts of the apparatus are a sample stage and two metal measurement tips.

Both the sample stage and the measurement tips are moved under computer control. For the electrical characterization, the stage is moved stepwise in a rectangular grid. At each grid point, the two measurement tips are lowered so as to form an electrical contact with the sample. The DC resistance measurement is carried out in a two-point arrangement with a multimeter (Multimeter 2000, Keithley Instruments), the inputs of which are connected to the two measurement tips. The respective measurement result is categorized. as positively conductive below 1 Mohm or evaluated as negative above 1 Mohm. The ratio between the number of positive measurements and the total number of measurements defines the normalized conductance as the measurement quantity to be taken into consideration.

The results of the DC resistance measurements are collated in Table 1 below:

| | Norm. conductance [%] | Norm. conductance [%] |
|---|---|---|
| | Spot 1 | Spot 2 |
| Chip 1 | | |
| T sample | 100 | 100 |
| C sample | 0 | 0 |
| Chip 2 | | |
| T sample | 0 | 0 |
| C sample | 0 | 0 |

The results demonstrate that very good discrimination between the alleles is achieved by labeling the target nucleic acid before the discrimination (chip 1). The absolute signal is furthermore increased on the “match” spot; only by virtue of this is electrical detection of the nucleic-acid hybridization possible at all under the selected conditions. Accordingly, a signal is not detectable on chip 2 for either one allele or the other.

### Example 2

### Analysis of the PCR Products of Patient Samples

The DNA of 19 different patients was studied by means of genotyping methods corresponding to the prior art (for example pyrosequencing) in respect of their CETP-Taq1 genotype. The DNA of the 19 different patients was then amplified by PCR using standard methods, a biotinylated primer and a non-biotinylated primer being used. The biotin primer had the sequence 5′-biotin- ttg tgt ttg tct gcg acc (SEQ ID NO: 3), and the sequence of the non-biotinylated primer was 5′-ccc aac acc aaa tat aca cca (SEQ ID NO: 4).

The biotinylated strand of the PCR product had the sequence:

5′-biotin- tt gtgtttgtct gcgacccaga atcactgggg ttcRagttag ggttcagatc tgagccaggt tagggggtta atgtcagggg gtaaagatta ggaggttggt gtatatttgg tgttggg (SEQ ID NO: 6); R=A or G. 19 DNA chips were prepared by immobilizing 5′-amino-modified, allele-specific oligonucleotides covalently on oxidized silicon chips, which were coated with polymers containing amine groups. The covalent immobilization was carried out by means of the homobifunctional cross-linker BS3 (bis-sulfo-succinimidyl suberate, from Pierce). The sequences of the allele-specific oligonucleotides were: 5′-amino- ttt ttt ttt cct aac tcg aac cc (SEQ ID NO: 1) (specific for G allele) and 5′-amino- ttt ttt ttt cct aac ttg aac cc (SEQ ID NO: 2) (specific for A allele). The chips had a size of 1 cm². The allele-specific oligonucleotides were immobilized on the chip surface in 5 μl duplicates, so that 4 spots were obtained per chip.

50 μl of the PCR products were in each case demineralized by centrifuging in Microcon-3 columns (from Millipore, MWCO=3,000). The demineralized PCR-products were dissolved in 45 μl of 0.11 N NaOH, 0.9 M NaCl, 0.005% SDS. The alkaline target solution was applied to the 19 DNA chips and incubated for 10 min at 25° C.

The alkaline hybridization solution was neutralized by adding 5 μl of 1 M NaH₂PO₄, 1 M NaCl, 0.005% SDS. The chips with the hybridization solutions were incubated for 15 h at 25° C. in a humid chamber (=non-stringent hybridization).

19×50 μl of a solution of streptavidin-gold (10 nm, Sigma) were centrifuged for 30 min at 21,000 g. The supernatants were removed and the residues were taken up in 0.1 M phosphate buffer (pH 8.2), 1 M NaCl, 0.005% SDS (=buffer A).

After the end of the non-stringent hybridizations, the chips were washed with buffer A and subsequently dried at 25° C. The chips were incubated for 2 h at 25° C., in each case with 50 μl of the streptavidin-gold solution. This was followed by discrimination of the alleles under stringent conditions. The discrimination was carried out by washing the chips for 5 min with preheated buffer A at 60° C. Buffer A was removed and washing was carried out with 0.1 M phosphate buffer (pH. 8.2), 1 M NaNO₃, 0.005% SDS in order to remove interfering chloride ions before the silver enhancement.

For the silver enhancement, a solution was prepared by mixing one part of an aqueous 0.012 M AgNO₃ solution and four parts of an aqueous solution of 0.05 M hydroquinone and 0.3 M sodium citrate buffer (pH 3.8). The chips were immersed in this solution for 9 min. The silver enhancement was ended by washing the chips with water.

The DC resistance measurement of the silver-enhanced chip surfaces was carried out between externally applied electrodes. Similarly as in Example 1, 25 measurements per DNA spot were carried out at different positions of the spot.

The results of the DC resistance measurements of the chips, which had been silver-enhanced for 9 min, are presented in Table 2. Those chips which still showed no signals after 9 min of silver enhancement were treated for a further 4 min. with the freshly prepared silver-nitrate/hydroquinone solution. After termination of the enhancement reaction by washing with water, the DC resistance measurement was carried out according to the method described above.

The results are presented in Table 2. 18 of the 19 genotypes were determined correctly. In one case (patient 13), a homozygotic G-allele-carrying patient was determined as a heierozygotic patient.

TABLE 2
Result of the analysis of the PCR products of patient samples

| | T-spot, 9′ | T-spot, 13′ | C-spot, 9′ | C-spot, 13′ | |
|---|---|---|---|---|---|
| | (norm. con- | (norm. con- | (norm. | (norm. | Reference |
| Patient | ductance [%]) | ductance [%]) | conductance [%]) | conductance [%]) | genotype |
| 16 | 100 | not measured | 0 | not measured | AA |
| 13 | 100 | not measured | 100 | not measured | GG |
| 5 | 0 | not measured | 20 | not measured | GG |
| 14 | 60 | not measured | 0 | not measured | AA |
| 12 | 100 | not measured | 0 | not measured | AA |
| 3 | 0 | 90 | 0 | 70 | AG |
| 6 | 0 | 100 | 0 | 50 | AG |
| 4 | 0 | 80 | 0 | 100 | AG |
| 2 | 0 | 35 | 0 | 50 | AG |
| 47 | 10 | not measured | 100 | not measured | GG |
| 45 | 10 | not measured | 100 | not measured | GG |
| 35 | 20 | not measured | 100 | not measured | GG |
| 32 | 0 | not measured | 100 | not measured | GG |
| 46 | 100 | not measured | 0 | not measured | AA |
| 44 | 100 | not measured | 0 | not measured | AA |
| 49 | 0 | 100 | 0 | 100 | AG |
| 50 | 0 | 100 | 0 | 100 | AG |
| 42 | 0 | 100 | 0 | 100 | AG |
| 41 | 0 | 100 | 0 | 100 | AG |

It should be understood that the preceding is merely a detailed description of a few embodiments of this invention and that numerous changes to the disclosed embodiments can be made in accordance with the disclosure herein without departing from the spirit or scope of the invention. The preceding description, therefore; is not meant to limit the scope of the invention. Rather, the scope of the invention is to be determined only by the appended claims and their equivalents.

1. Method for the detection of target nucleic acids from a mixture of different nucleic acids, said method comprising the steps of:
A) providing a surface having immobilized nucleic acids or nucleic-acid analogues, which are suitable for forming non-covalent (base-pair) bonds with the target nucleic acids,
B) non-stringent hybridization of the target nucleic acids to be detected onto the immobilized nucleic acids from a solution of the analyte nucleic acid,
C) labeling of the nucleic acids of the analysis mixture with labeling elements,
D) optionally repeated treatment of the surface with a washing liquid in order to remove weakly bound nucleic acids, and
E) detection of the nucleic-acid pairs remaining on the surface with the aid of the labeling unit bonded to them, by means of optical, optical-spectroscopic, electrical, mechanical or magnetic detection methods;
wherein steps B) and C) can be carried out in any order.

2. Method according to claim 1, comprising the steps of:
A) providing a surface having immobilized nucleic acids or nucleic-acid analogues, which are suitable for forming non-covalent (base-pair) bonds with the target nucleic acids,
B) non-stringent hybridization of the target nucleic acids to be detected onto the immobilized nucleic acids from a solution of the analyte nucleic acid,
C) labeling of the nucleic acids of the analysis mixture with labeling elements,
D) optionally repeated treatment of the surface with a washing liquid in order to remove weakly bound nucleic acids, and
E) detection of the nucleic-acid pairs remaining on the surface with the aid of the labeling unit bonded to them, by means of optical, optical-spectroscopic, electrical, mechanical or magnetic detection methods.

3. Method according to claim 1, comprising the steps of:
A) providing a surface having immobilized nucleic acids or nucleic-acid analogues, which are suitable for forming non-covalent (base-pair) bonds with the target nucleic acids,
B′) labeling of the nucleic acids of the analysis mixture with labeling elements,
C′) non-stringent hybridization of the labeled nucleic acids onto the immobilized nucleic acids,
D) optionally repeated treatment of the surface with a washing liquid in order to remove weakly bound nucleic acids, and
E) detection of the nucleic-acid pairs remaining on the surface with the aid of the labeling unit bonded to them, by means of optical, optical-spectroscopic, electrical, mechanical or magnetic detection methods.

4. Method according to claim 1, wherein the washing steps are stringent, and are carried out with thermally regulated buffer solutions, the temperature of the buffer solution lying above the temperature selected for the hybridization of the analyte nucleic acid onto the immobilized nucleic acids.

5. Method according to claim 1, wherein the washing steps are stringent, and are carried out with buffer solutions; the ionic strength of the buffer solution lying below the analyte-solution ionic strength selected for the hybridization of the analyte nucleic acid onto the immobilized nucleic acids.

6. Method according to claim 5, wherein the stringent washing steps are carried out with at least one sodium-chloride buffer solution, the concentration of the buffer solution(s) lying below the sodium-chloride concentration selected for the hybridization onto the immobilized nucleic acids.

7. Method according to claim 1, wherein the nucleic acid to be detected is coupled to ligands which bind to ligand-binding receptor molecules, with which the labeling units were linked or coated.

8. Method according to claim 7, wherein the receptor is selected from the group consisting of avidin, neutravidin and streptavidin, and the ligand is biotin.

9. Method according to claim 7, wherein the receptor is an antibody and its antigen is the ligand.

10. Method according to claim 1, wherein the linking of the target nucleic acid with ligands is carried out by enzymatic or chemical methods or by intercalation.

11. Method according to claim 1, wherein the labeling units are nanoparticles, metal complexes and/or clusters based on elements selected from the group consisting of Au, Ag, Pt, Pd and C.

12. Method according to claim 1, wherein the labeling units have a molecular weight of more than 10,000 g/mol.

13. Method according to claim 1, wherein the labeling units are enhanced before or during the detection E).

14. Method according to claim 1, wherein the surface has a set of different immobilized nucleic acids or nucleic-acid analogues.

15. Method according to claim 1, wherein the nucleic acid to be detected is linked with biotin as a ligand, and the labeling is carried out using gold particles coated with avidin, neutravidin or streptavidin as a receptor.

16. Method according to claim 1, wherein the nucleic acid to be detected is linked with an antigen, and the labeling is carried out using gold particles coated with antibodies.

17. Method according to claim 15, wherein the gold particles are enhanced by an autometallographic reaction and the detection of the nucleic acid is carried out optically or electrically.

18. Method according to claim 16, wherein the gold particles are enhanced by an autometallographic reaction and the detection of the nucleic acid is carried out optically or electrically.

19. Method according to claim 1, wherein the target nucleic acids are detected for the purpose of the expression profiling of ribonucleic acids, or for the analysis of single point mutations (SNPs) or for the analysis of amplified genes.


SEQUENCE LISTING (6 sequences)

SEQ ID NO: 1 - DNA, 23 bp, Artificial
  Description of Artificial Allele-Specific Sequence
tttttttttcctaactcgaaccc

SEQ ID NO: 2 - DNA, 23 bp, Artificial
  Description of Artificial Allele-Specific Sequence
tttttttttcctaacttgaaccc

SEQ ID NO: 3 - DNA, 17 bp, Artificial
  Description of artificial biotinylated primer sequence
ttgtgttgtctgcgacc

SEQ ID NO: 4 - DNA, 21 bp, Artificial
  Description of artificial biotinylated primer sequence
cccaacaccaaatatacacca

SEQ ID NO: 5 - DNA, 119 bp, Artificial
  Description of Artificial PCR Product Sequence
ttgtgtttgtctgcgacccagaatcactggggttcaagttagggttcagatctgagccaggttagggggttaatgtcagggggtaaagattaggaggttggtgtatatttggtgttggg

SEQ ID NO: 6 - DNA, 119 bp, Artificial
  Description of Artificial PCR Product Sequence
ttgtgtttgtctgcgacccagaatcactggggttcragttagggttcagatctgagccaggttagggggttaatgtcagggggtaaagattaggaggttggtgtatatttggtgttggg